SEQUENCE LISTING (10 sequences)

SEQ ID NO: 1 - DNA, 19 bp, Artificial Sequence
  Description of Artificial Sequence Synthetic oligonucleotide
cacggctcgcggcgcttta

SEQ ID NO: 2 - DNA, 19 bp, Artificial Sequence
  Description of Artificial Sequence Synthetic oligonucleotide
cgtctatggagtcactgtg

SEQ ID NO: 3 - DNA, 55 bp, Artificial Sequence
  Description of Artificial Sequence Synthetic oligonucleotide
gatcccacggctcgcggcgctttattcaagagataaagcgccgcgagccgtgaaa

SEQ ID NO: 4 - DNA, 55 bp, Artificial Sequence
  Description of Artificial Sequence Synthetic oligonucleotide
agcttttcacggctcgcggcgctttatctcttgaataaagcgccgcgagccgtgg

SEQ ID NO: 5 - DNA, 55 bp, Artificial Sequence
  Description of Artificial Sequence Synthetic oligonucleotide
gatcccgtctatggagtcactgtgttcaagagacacagtgactccatagacgtta

SEQ ID NO: 6 - DNA, 55 bp, Artificial Sequence
  Description of Artificial Sequence Synthetic oligonucleotide
agcttaacgtctatggagtcactgtgtctcttgaacacagtgactccatagacgg

SEQ ID NO: 7 - DNA, 19 bp, Homo sapiens
ggtgggattctccctgttg

SEQ ID NO: 8 - DNA, 19 bp, Homo sapiens
gcgagccatggctgtctac

SEQ ID NO: 9 - DNA, 20 bp, Artificial Sequence
  Description of Artificial Sequence Synthetic primer
cccagagtttctgtggctct

SEQ ID NO: 10 - DNA, 20 bp, Artificial Sequence
  Description of Artificial Sequence Synthetic primer
ggacacttcagccagcaaat

(19) United States
(12) Patent Application Publication
(10) Pub. No.: US 2011/0269815 A1
(43) Pub. Date: Nov. 3, 2011

(54) METHODS OF REDUCING CELLULAR PROLIFERATION BY INHIBITING ACSVL3

(75) Inventors: Paul A. Watkins, Glen Arm, MD (US); John Laterra, Baltimore, MD (US)

(73) Assignee: THE JOHNS HOPKINS UNIVERSITY, Baltimore, MD (US)

(21) Appl. No.: 13/001,066

(22) PCT Filed: Jun. 25, 2009
(86) PCT No.: PCT/US2009/048705

§ 371 Date: Dec. 23, 2010

Related U.S. Application Data
(60) Provisional application No. 61/133,064, filed on Jun. 25, 2008.

(51) Int. Cl. A61K 31/7088; C07H 21/02; A61K 31/497; A61K 31/5415; C12N 5/09; A61P 35/00

(52) U.S. Cl. 514/44 A; 435/375; 435/366; 514/252.13; 514/226.2; 514/225.2; 536/24.5

(57) ABSTRACT

The present invention is directed to methods of reducing cancer cell proliferation and/or treating cancer by administering a therapeutically effective amount of a compound that inhibits the activity of ACSVL3.

### STATEMENT OF GOVERNMENT RIGHTS

This invention was made with U.S. Government support under grants NS037355 (PAW), NS043987 (JL), and CA129192 (JL) awarded by the National Institutes of Health. The Government has certain rights in this invention.

### BACKGROUND

Many human cancers are refractory to conventional therapeutic approaches. Therefore, identification of promising new molecular targets and therapeutics is needed. We have identified an enzyme (ACSVL3) involved in lipid (fat) metabolism that is expressed at unusually high levels in many human cancers. In some cancers, this enzyme is not normally present in the cell type of origin of the tumor, yet is abundant when these cells become malignant. Human cancer cell lines that exhibit malignant behavior in culture were found to have a less malignant phenotype when depleted of this enzyme. Human cancer cell lines that produce rapidly growing malignant tumors when injected into mice generated either no tumors or slower growing tumors when cells were first depleted of this enzyme. Certain cell signaling pathways known to be required for the malignant phenotype are disrupted when cells are depleted of this enzyme. Based on these observations, we conclude that this enzyme is a new target for human cancer therapy.

For example, gliomas account for more than 50% of all primary brain tumors, and nearly two-thirds of gliomas are highly aggressive with “malignant” pathological features (WHO Grade III or IV) (1). Despite advances in the neurosurgical, radiotherapeutic, and chemotherapeutic treatment of gliomas, the prognosis generally remains poor (1).

A high rate of lipid synthesis is needed to support membrane biogenesis required for tumor growth. Lipids also play key roles in second messenger pathways that are dysregulated in malignant cells, and elevations in specific lipid messengers are associated with malignancy (2). The primary source of the fatty acid (FA) constituents of tumor lipids is de novo synthesis and not uptake from extracellular sources; thus, cancer cells typically have increased rates of FA synthesis (3). The observation that a breast cancer biomarker, OA-519, was identical to a key enzyme in the FA synthesis pathway, fatty acid synthase (FASN) (4), inspired renewed interest in tumor cell lipid metabolism. Subsequently, high FASN levels have been found in many human cancers, including primary brain cancer (5, 6), and this finding is associated with a poor prognosis (4).

The product of FASN is a 16-carbon FA. Newly synthesized FAs, as well as FAs containing any number of carbon atoms and derived from any source, must be “activated” by thioesterification to coenzyme A (CoA) for further metabolism. Acyl-CoA synthetases (ACS) catalyzing this reaction thus play a central role in providing activated FAs for complex lipid synthesis, energy-yielding catabolic pathways, protein acylation, and other processes (7). Humans have 26 genes encoding ACSs that differ in their ability to activate short-, medium-, long-, and very-long-chain FAs (8). The six-member very-long-chain ACS (ACSVL) family includes ACSVL3 (SLC27A3; also known as fatty acid transport protein (FATP) 3), an ACS that activates saturated FAs containing 16 to 24 carbons (9). The biological basis for the numerous ACS enzymes is only partially understood. Reasons in addition to their distinct FA substrate specificities include restricted expression to specific cell types and restricted targeting to specific subcellular organelles. In the adult mouse brain, ACSVL3 protein is detectable at low levels in neurons but not glia (9).

We have discovered that human malignant gliomas abundantly overexpress ACSVL3, and that depletion of this enzyme decreases the malignant phenotype of glioma cells in vitro, and of glioma xenografts in mice. These effects are mediated, in part, by changes in RTK-dependent signaling via Akt. Approaches for treating cancer and reducing cancer cell proliferation based on these discoveries are disclosed herein.

### SUMMARY

Embodiments of the invention described herein are directed to compounds (e.g., siRNA) for and methods of reducing cancer cell proliferation by administering an effective amount of a compound that inhibits the activity of ACSVL3 to a cancer cell expressing ACSVL3. For example, an effective amount of a specific siRNA, flupenthixol, triflupromazine, perphenizine, or chlorpromazine may be used.

Embodiments are also directed to methods of treating cancer by administering a therapeutically effective amount of a compound that inhibits the activity of ACSVL3 to a cancer cell expressing ACSVL3. For example, administering compounds to treat cancerous glioma cells or human bronchial epithelial cells.

This application claims priority to U.S. Application 61/133,064 filed Jun. 25, 2008, the contents of which are incorporated herein in their entirety.

### BRIEF DESCRIPTION OF FIGURES

FIG. 1 shows expression of ACSVL3 in normal human brain and in malignant gliomas. A tissue array containing biopsies from 79 different malignant gliomas, including 19 astrocytomas, 16 oligodendrogliomas, 19 anaplastic astrocytomas, 19 glioblastomas multiforme, and 6 of mixed etiology, and sections of normal human brain (all present in quadruplicate) was obtained. Immunohistochemical staining of normal brain (A, brown stain) shows ACSVL3 expression mainly in neurons (arrows) but not glia. Increased ACSVL3 expression was observed in all gliomas examined. Representative astrocytoma (B), oligodendroglioma (C), anaplastic astrocytoma (D), and glioblastoma multiforme (E) are shown. F-J. ACSBG1 expression. ACSBG1 is also found in neurons (arrows) in normal human brain (F) but is not increased in gliomas (G-J). Vertical pairs (AF, BG, CH, DI, EJ) represent consecutive slices from the same biopsy.

FIG. 2 shows ACSVL3 expression in cultured human glioma cells and xenografts correlates with RTK activation. A. Human U87, U373, and Mayo-22 glioma cells were grown to ˜80% confluence. Cells were either untreated or incubated 16 hrs in the presence of HGF (20 ng/ml; gift from Genentech; South San Francisco Calif.) or EGF (30 ng/ml; Calbiochem/EMD; San Diego Calif.) before harvesting and Western blot analysis for ACSVL3 expression. Each lane contained 30 μg total cellular protein. Actin was detected as a loading control. The three sections were from the same Western blot; images were obtained simultaneously. B. U87 cells stably expressing constitutively active EGFR (EGFRvIII) or corresponding control U87 cells were harvested and analyzed by Western blot. Actin was detected as a loading control. C. Tumors were produced in the flanks of athymic nude mice (n=12) by s.c. injection (2 sites per mouse) of 4×10⁶ U87 cells. When tumors size reached ˜300 mm³ (day 14), animals were randomly divided into two groups of 6 mice. One group received i.p. injection (100 μg/20 g body wt) of anti-HGF mAb (L2G7) and the other group received isotype control mAb (5G8) on days 14, 16, and 18. Mice were sacrificed on day 20. Tumor homogenates (50 μg protein) were subjected to Western blot analysis for ACSVL3 expression. Actin expression was used as a loading control.

FIG. 3 shows a stable knockdown of ACSVL3 in U87 glioma cells. Clones with stable KD of ACSVL3 and appropriate controls were produced using shRNA plasmids ACSVL3-3 and ACSVL3-4, alone or together, as described in Methods. ACSVL3 expression was determined by indirect immunofluorescence analysis (A) or by Western blot (B). Immunofluorescence images were captured using identical exposure times. For Western blots, each lane contained 30 mg total cellular protein. Actin was detected as a loading control.

FIG. 4 shows growth properties of control and ACSVL3 KD glioma cells. A & B, U87 cells. A. Growth in culture of two control U87 cell lines (scrambled shRNA), two ACSVL3 KD lines (one generated from the ACSVL3-3 shRNA plasmid and the other from ACSVL3-4), one ACSF2 KD line, and one FATP4 KD line were measured. Cells (5000 cells/well) were seeded into 6-well plates; at the indicated times, cells from triplicate wells were harvested and each was counted in duplicate using a hemacytometer. B. Anchorage-independent growth of control, ACSVL3-3 KD, and FATP4 KD U87 cells. Cells (5000 cells/well) were mixed with soft agar and seeded into 6-well plates. On day 20, cells from triplicate wells were examined microscopically for colony formation. C&D, Mayo-22 cells. Cells were transiently transfected with either ACSVL3-3 siRNA or control (scrambled) siRNA as described in Methods. Anchorage-dependent growth was measured on day 7 following transfection (C) and anchorage-independent growth was measured on day 18 post-transfection (D) as described above for U87 cells. For all panels, mean±standard error is shown. Statistical significance was determined by one-way ANOVA with Bonferroni's multiple comparison test (panels A and B) or Student's t-test (panels C and D). ***, p<0.001; **, p<0.01; n.s., p>0.05.

FIG. 5 shows in vivo tumorigenesis of ACSVL3 KD U87 cells. A-C, subcutaneous xenografts. Either control or KD ACSVL3-3 U87 cells (4×10⁶) were injected s.c. in the flanks of athymic nude mice (2 sites/mouse; 6 mice/group). A. Tumorigenicity. Tumors were produced at all 12 sites for control cells but only at 7 of 12 injection sites (58%) for ACSVL3 KD cells. B. Tumor growth rates. Tumor length and width were measured using calipers and tumor volume was calculated using the formula volume=(length×width²)/2. C. Western blots of xenograft homogenates verifying ACSVL3 knockdown. Each lane contained 50 μg total protein. Three randomly chosen control tumors were compared to the seven ACSVL3 KD tumors. D. Intracranial xenografts. Groups of 5 mice received either control, KD ACSVL3-4, or KD ACSVL3-3+ACSVL3-4 U87 cells (10⁵) injected into the right caudate/putamen; on day 26 post-injection, animals were sacrificed and tumor volume was calculated as described in Methods. For panel B, mean±standard error is shown; for panel D, mean±standard deviation is shown. Statistical significance was determined by Student's t-test (panel B) or one-way ANOVA with Bonferroni's multiple comparison test (panel D). ***, p<0.001 vs. control.

FIG. 6 shows Akt signaling in control and ACSVL3 KD U87 cells. A & B. Effect of HGF treatment on Akt phosphorylation and stability. Control and ACSVL3-3 KD U87 cells were incubated with HGF (20 ng/ml) for the indicated time prior to analysis for total Akt and phospho-Akt levels by Western blot. A, LiCOR Odyssey images; control and KD images were obtained simultaneously. B, Quantitation of the LiCOR data. C. Effect of caspase inhibition on Akt stability. One control and two different knockdown clones produced using the ACSVL3-3 shRNA plasmid were incubated with HGF (20 ng/ml) for the indicated time in the absence or presence of the pan-caspase inhibitor, Z-VAD-FMK (50 μM), prior to analysis for total Akt and phospho-Akt levels by Western blot. D. Constitutively active Akt reverses the growth-inhibitory effects of ACSVL3 KD. Control or KD U87 cells were transfected with myrAkt (gift of Dr. M. E. Greenberg, Harvard Medical School) or empty vector using Nucleofector Kit T with the Nucleofector™ apparatus (amaxa; Cologne, Germany). Left, adherent growth was measured 11.5 days post-transfection as described in the legend to FIG. 4A. Right, anchorage-independent growth was measured by colony formation in soft agar on day 17 post-transfection as described in the legend to FIG. 4B. Mean±standard error is shown; statistical significance was determined by one-way ANOVA with Bonferroni's multiple comparison test. ***, p<0.001 vs. control vector; **, p<0.01 vs. control vector; n.s., p>0.05 vs. control myr-Akt.

FIG. 7 shows expression of ACSVL3 in normal mouse brain. Immunohistochemistry for ACSVL3 expression (brown stain) revealed that the protein was expressed mainly in neurons, not glia.

FIG. 8 shows the ACSVL3 protein is upregulated in lung cancer cell lines. Cells were cultured in either synthetic medium or serum-containing medium for at least 2 generations before harvesting for immunoblot analysis for ACSVL3

FIG. 9 shows neutral lipid synthesis in U87 cells. Control and ACSVL3 KD U87 cell were incubated with [1-14C]C16:0 and neutral lipid classes were resolved by TLC. Phosphorimager analysis of TLC plates, along with the positions of authentic standards, is shown. Label incorporation into DAG was reduced in ACSVL3 KD cells.

FIG. 10 shows Akt phosphorylation in xenografts. Homogenates of 4 tumors generated from control U87 cells and 4 from ACSVL3 knockdown (KD) cells were analyzed for total and phospho-Akt by Western blotting; actin was used as a loading control.

FIG. 11 shows ACSVL3 mRNA levels were significantly greater in glioblastomas (G1-G5) than in normal brain (N1, N2).

FIG. 12 shows a Western blot demonstrating that ACSVL3 protein levels are increased in U373 cells as early as 2 hours following treatment with HGF.

FIG. 13 shows that in the absence of HGF, neither actinomycin D nor cycloheximide affected ACSVL3 expression. When HGF was added, both actinomycin D and cycloheximide prevented the increase in ACSVL3 expression seen in the absence of these agents.

FIG. 14 shows a comparison test for compounds that inhibit ACSVL3-containing U87 cells (gray bars) but not ACSVL3-deficient U87 cells (white bars).

FIG. 15 shows ACSVL3 expression in normal and cancerous human lung. A tissue array containing samples from 69 lung tumors and 3 sections of normal lung was examined for ACSVL3 expression (brown stain) by immunohistochemistry. The slide was counterstained with hematoxylin (blue stain). A. Normal lung showing weak ACSVL3 staining (arrows). B-D. Three representative lung tumors.

FIG. 16 shows growth in culture of control and ACSVL3 KD lung tumor cells. Control (CON) and ACSVL3 knockdown (KD) H460 (top) or H82 (bottom) cells were seeded into 6-well plates (5000 cells/well). On the indicated days, cells from triplicate wells were harvested and counted in duplicate using a hemacytometer. Mean±SD is plotted.

FIG. 17 shows anchorage-independent growth of control and ACSVL3 KD lung tumor cells. CON and ACSVL3 KD A549, EKVX, H460, and H82 lung tumor cells were mixed with soft agar and seeded into 6-well plates (5000 cells/well). On day 20, cells from triplicate wells were examined microscopically for colony formation. Data from the two controls and the two ACSVL3 KD lines were combined. Mean±SD is plotted. The mean percent decrease in colony formation for each line is indicated.

### DETAILED DESCRIPTION

The invention described herein deals with the identification of a new target for cancer therapeutics and compounds effective in reducing cancer cell proliferation. Specifically, expression of the fatty acyl-CoA synthetase, ACSVL3, was found to be markedly elevated in clinical malignant glioma specimens but nearly undetectable in normal glia. ACSVL3 is identical to the protein also known as Fatty Acid Transport Protein 3 (FATP3), and is the product of the gene annotated as SLC27A3 in databases maintained by the National Center for Biotechnology Information (NCBI). The Gene identification (ID) number for human SLC27A3 is 11000.

ACSVL3 levels correlated with the malignant behavior of human glioma cell lines and glioma cells propagated as xenografts. ACSVL3 expression was induced by the oncogenic receptor tyrosine kinases (RTK) c-Met and EGFR. Treating HGF-dependent tumor xenografts with neutralizing anti-HGF monoclonal antibodies reduced ACSVL3 expression concurrent with tumor growth inhibition in vivo. ACSVL3 expression knockdown using RNA interference decreased activation of long- and very-long-chain fatty acids and inhibited anchorage-dependent and anchorage-independent glioma cell growth by ˜70% and ˜90%, respectively. ACSVL3-depleted cells were less tumorigenic than control cells and subcutaneous xenografts grew ˜60% slower than control tumors. Orthotopic xenografts produced by ACSVL3-depleted cells were 82-86% smaller than control xenografts. ACSVL3 knockdown disrupted Akt function as evidenced by an RTK-induced transient loss of both total and phosphorylated Akt via a caspase-dependent mechanism. Expressing constitutively active myr-Akt rescued cells from the anchorage-dependent and anchorage-independent growth inhibitory effects of ACSVL3 depletion. These studies, which are presented in more detail in the Examples, show that ACSVL3 maintains oncogenic properties of malignant glioma cells via a mechanism that involves, at least in part, the regulation of Akt function.

Brain malignancies are responsible for significant morbidity and mortality in both adults and children. Despite advances in surgical, drug, radiation, and combination therapies, median survival rates for adults with glioblastoma remain less than two years and primary brain tumors are currently the leading cause of cancer-related death in children. Lipogenic and fatty acid synthetic pathways are hyper-activated in rapidly growing cancer cells (3) and several studies indicate that targeting enzymes in FA and lipid metabolic pathways may be of therapeutic value in human malignancies. Increased expression of fatty acid synthase, an enzyme required for FA synthesis, has been reported in many human tumors including brain tumors (5). High levels of FASN protein occur in rat and human glioma cell lines, and in human glioma tissue samples (6), and FASN inhibitors show significant antitumor effects both in vitro and in xenografts (24).

Because of their central position in FA metabolism, ACSs are also rational choices for investigation as therapeutic targets. Unlike FASN, the only known fatty acid synthase, the 26 known ACSs reflect significant metabolic complexity at the level of fatty acid activation (8). This complexity is due to known differences in ACS substrate specificities and other less understood functions such as enzyme-specific targeting to support specialized subcellular lipogenic requirements. We show herein that ACSVL3, an enzyme not normally found in glia, is expressed at extraordinarily high levels in human malignant gliomas. We also found that ACSVL3 levels were elevated in tumorigenic glioma cells and that expression levels correlated with more aggressive tumorigenic phenotypes. We previously found that ACSVL3 mRNA is high in embryonic brain and decreases to very low levels in adult brain (9). Together, these findings suggested that ACSVL3 might function during periods of mitogenic pathway activation and rapid cell growth, and that depleting cells of this enzyme could have anti-tumor effects. Our findings that ACSVL3 depletion inhibits glioma cell tumorigenicity and the growth rates of glioma cell lines and glioma xenografts support this hypothesis. To the best of our knowledge, this report is the first to implicate an ACSVL family member in human cancer.

Distinct from our findings with ACSVL3, we show that the glioma malignant phenotype is not associated with or dependent upon at least three other ACS family members. Specifically, ACSBG1 expression was not found to be increased in clinical glioma specimens, and knockdown of either FATP4 or ACSF2 did not ameliorate the malignant phenotype of glioma cells in vitro. It remains possible that other ACSs may contribute to the malignancy of glioma or other cancers. While few studies of ACSs in human cancers have been reported, most have focused on a long-chain ACS family member, ACSL5, the expression of which appears to be variably associated with malignancy. ACSL5 expression was found to be increased in many colorectal tumors (25, 26). Some small intestinal tumors including adenocarcinomas had decreased levels (26). ACSL5 expression was also increased in well-differentiated, but not poorly-differentiated, endometrial adenocarcinomas (27). ACSL5 mRNA levels were high in primary human gliomas and A172 glioma cells but, in contrast to our findings with ACSVL3, were not detectable in either U87 or U373 glioma cells (28). To our knowledge, gain- or loss-of function studies directed at determining to role of ACSL5 in the cancer phenotype have not been reported.

The methods disclosed herein can involve administering a compound that inhibits or otherwise prevents the activity of the ACSVL3. Few inhibitors of the ACS reaction are known. The most studied is triacsin c, a non-specific compound that inhibits ACSL1, 3, and 4, and ACSVL1 (FATP2) but does not inhibit ACSL5, ACSL6, ACSVL5 (FATP4), or ACSBG2 (29-34). The effect of triacsin c on ACSVL3 activity is not known. Mashima et al. reported that triacsin c induced caspase activity in human lung (NCI-H23), colon (HCT-15), and brain (SF268) cancer cell lines (35). The mitochondrial level of cardiolipin, an inner membrane lipid which is important for retaining cytochrome c, was decreased by triacsin c treatment suggesting this as a possible mechanism by which ACS inhibition promotes apoptosis. While we observed little cell death in response to ACSVL3 knockdown, our data showing a caspase-mediated Akt-dependent mechanism by which ACSVL3 knockdown alters glioma cell growth may reflect the observations by Mashima et al.

Overactivation of tumor receptor tyrosine kinases (e.g. EGFR, PDGF, c-Met) and their downstream signaling pathways is closely associated with malignant progression and poor patient survival (22, 36-39). Our findings demonstrate a relationship between ACSVL3 expression, functional RTK signaling and the malignant phenotype in the human glioma models examined. The most tumorigenic models examined (U87, Mayo-22) expressed much higher basal levels of ACSVL3 compared to the less tumorigenic U373 glioma cells. Activating c-Met and expressing EGFRvIII, which enhance glioma cell malignancy by multiple criteria, were found to induce ACSVL3 expression (11, 13). Conversely, inhibiting c-Met signaling in animals bearing glioma xenografts was associated with ACSVL3 expression inhibition concurrent with tumor regression. RTKs such as c-Met exert their oncogenic effects via multiple downstream signaling pathways among which Akt activation (i.e. phosphorylation) by PI3K plays a prominent role (40). Depleting glioma cells of ACSVL3 was found to significantly alter Akt function as evidenced by a rapid but transient decrease in both the phosphorylated and non-phosphorylated forms of Akt in response to either c-Met or EGFR agonists. The caspase-dependency of this activation-induced aberrant Akt response is consistent with a proteolytic mechanism (41, 42). While it is well known that Akt phosphorylation requires protein translocation to membrane, details regarding the phospholipids involved are currently undefined. Our findings suggests that ACSVL3 enzymatic activity is necessary to maintain phospholipid membrane specializations required for optimal Akt stability. Possibilities include production of lipidic signaling molecules such as specific phosphoinositide species and diacylglycerol, or the generation of acylated proteins known to participate in RTK signaling pathways. That expression of constitutively active Akt reversed the glioma growth inhibiting effects of ACSVL3 knockdown strongly supports a functional interaction between ACSVL3-dependent metabolic functions and Akt-dependent oncogenic signaling. Identifying the precise biochemistry by which ACSVL3 supports the malignant phenotype and Akt function should provide new insights into the interrelationships between fatty acid metabolism and molecular oncogenesis.

In some embodiments, the methods disclosed herein can be directed to the administration of a compound or compounds designed to inhibit the activity of ACSVL3. In some embodiments, the method is directed to reducing cancer cell proliferation by administering a compound that inhibits the activity of ACSVL3 to a cancer cell expressing ACSVL3.

The compound administered can be any small or large molecule found to inhibit or otherwise interfere with the activity of ACSVL3. For example, the compound can be selected from the group consisting of flupenthixol, triflupromazine, perphenizine, and chlorpromazine. As one of skill in the art will appreciate, related compounds belonging to the thiothixene family of antipsychotic drugs and/or related members of the phenothiazine family of antipsychotic drugs may also be used in the methods. Other suitable compounds such as antibodies, siRNA, or other compounds can also be administered. In some embodiments, the compounds exhibit a high degree of specificity for binding with ACSVL3 or otherwise interfering with its activity.

The cells in the methods disclosed can be any cell expressing ACSVL3. The cells can express normal levels of this enzyme or in some embodiments the cells can overexpress ACSVL3. The cells can be, for example, cancer cells such as glioma cells or human lung cancer cells. However, one of skill in the art will appreciate that other cancer cells may be treated using the methods disclosed therein.

The methods disclosed herein can reduce the proliferation of cancer cells in vitro or in vivo. For example, the reduction of cellular proliferation can be from about 100% to about 1%, from about 95% to about 10%, from about 20% to about 90%, or other significant reduction. The term “about” as used herein indicates plus or minus ten percent of the number it is used in conjunction with.

A wide array of patients can benefit from the methods disclosed herein. Persons or other mammals suffering from cancers that overexpress or express ACSVL3 can be in need of these treatments, for example, humans with gliomas. The cancer cells can be aggregates of cells such as tumors or individual cells, for example, cells that may be systemically dispersed in the blood. The methods used herein can also be used in combination with other cancer therapeutics.

The methods of the invention can involve administering pharmaceutical compositions that are useful in the methods herein prepared with a therapeutically effective amount of a compound of the invention, as defined herein, and a pharmaceutically acceptable carrier or diluent.

The compounds of the invention can be formulated as pharmaceutical compositions and administered to a subject in need of treatment, for example a mammal, such as a human patient, in a variety of forms adapted to the chosen route of administration, for example, orally, nasally, intraperitoneally, or parenterally, by intravenous, intramuscular, topical or subcutaneous routes, or by injection into tissue.

Suitable oral forms for administering the compounds include, lozenges, troches, tablets, capsules, effervescent tablets, orally disintegrating tablets, floating tablets designed to increase gastric retention times, buccal patches, and sublingual tablets.

The compounds of the invention may be systemically administered, e.g., orally, in combination with a pharmaceutically acceptable vehicle such as an inert diluent or an assimilable edible carrier, or by inhalation or insufflation. They may be enclosed in coated or uncoated hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's), which is herein incorporated by reference in its entirety.

The compounds may be combined with a fine inert powdered carrier and inhaled by the subject or insufflated. Such compositions and preparations should contain at least 0.01% compounds. The percentage of the compositions and preparations may, of course, be varied and may conveniently be between about 2% to about 60% of the weight of a given unit dosage form. The amount of compounds in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. A syrup or elixir may contain the active compound, sucrose or fructose as a sweetening agent, methyl and propylparabens as preservatives, a dye and flavoring such as cherry or orange flavor.

Various other materials may be present as coatings or to otherwise modify the physical form of the solid unit dosage form. For instance, tablets, pills, or capsules may be coated with gelatin, wax, shellac or sugar and the like. Of course, any material used in preparing any unit dosage form should be pharmaceutically acceptable and substantially non-toxic in the amounts employed.

In addition, the compounds may be incorporated into sustained-release preparations and devices. For example, the compounds may be incorporated into time release capsules, time release tablets, and time release pills. In some embodiments, the composition is administered using a dosage form selected from the group consisting of effervescent tablets, orally disintegrating tablets, floating tablets designed to increase gastric retention times, buccal patches, and sublingual tablets.

The compounds may also be administered intravenously or intraperitoneally by infusion or injection. Solutions of the compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds disclosed for use in the methods herein can be determined by comparing their in vitro activity, and in vivo activity in animal models. Methods for the extrapolation of effective dosages in mice, and other animals, to humans are known to the art; for example, see U.S. Pat. No. 4,938,949, which is hereby incorporated by reference.

For example, the concentration of the compounds in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

The amount of the compounds required for use in treatment will vary not only with the particular salt selected but also with the route of administration, the nature of the condition being treated and the age and condition of the patient and will be ultimately at the discretion of the attendant physician or clinician.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

In some embodiments, the pharmaceutical compositions described herein contain a therapeutically effective dose of the compound. The term “effective amount” or “therapeutically effective amount,” as used herein, refers to the amount of the active compound that is effective to achieve its intended purpose after a single dose, wherein a single dose comprises one or more dosage units, or after a course of doses, e.g., during or at the end of the treatment period. The therapeutically effective amount will vary depending on the needs of the subject, but this amount can readily be determined by one of skill in the art, for example, a physician.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose will be in the range of from about 0.001 to about 100 mg/kg, e.g., from about 0.01 to about 100 mg/kg of body weight per day, such as above about 0.1 mg per kilogram, or in a range of from about 1 to about 10 mg per kilogram body weight of the recipient per day. For example, a suitable dose may be about 1 mg/kg, 10 mg/kg, or 50 mg/kg of body weight per day.

The compounds are conveniently administered in unit dosage faun; for example, containing 0.05 to 10000 mg, 0.5 to 10000 mg, 5 to 1000 mg, or about 100 mg of active ingredient per unit dosage foam. In some embodiments, the dosage unit contains about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 750 mg, or about 1000 mg of active ingredient.

The compounds may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

### Example 1

### Materials and Methods

Cell culture. Human U87 and U373 glioblastoma cell lines (American Type Culture Collection, Rockville, Md.) were cultured as previously described (10). U87 cells stably expressing EGFRvIII (11), and corresponding control line were from Dr. Gregory Riggins, Johns Hopkins University School of Medicine. Mayo 22 cells were a gift from Dr. C. David James (University of California San Francisco) and were maintained by serial passage as subcutaneous xenografts as described (12); for study, tumor cells were dissociated from resected xenografts and cultured for up to five passages. Cell proliferation, and anchorage-independent growth, and tritiated thymidine incorporation were measured as previously described (13, 14).

Transient ACSVL3 KD. Four different small interfering RNA (siRNA) constructs targeting different regions of ACSVL3 mRNA were produced using the pSilencer™ kit (Applied Biosystems/Ambion; Austin Tex.). U87 cells were transfected with each construct using siPORT™ Lipid reagent (Applied Biosystems/Ambion). ACSVL3 expression was assessed on day 3 post-transfection by indirect immunofluorescence and Western blot and found to be decreased by siRNA ACSVL3-3 and -4, but not -1 or -2 (data not shown). siRNA ACSVL3-3 (5′-CACGGCTCGCGGCGCTTTA-3′ (SEQ ID NO: 1)) targets by 394-412 of ACSVL3 mRNA and ACSVL3-4 (5′-CGTCTATGGAGTCACTGTG-3′ (SEQ ID NO: 2)) targets by 1861-1879. Transient ACSVL3 KD in Mayo-22 cells was achieved by transfection with ACSVL3-3 using siPORT™ Lipid reagent. Control cells received siRNA with a scrambled nucleotide sequence (supplied by Ambion).

Production of stable KD cell lines. ACSVL3-3 and -4 siRNA sequences were used to construct short hairpin RNA (shRNA)-producing vectors. Oligonucleotides (Integrated DNA Technologies; Coralville Iowa) 5′-GATCCCACGGCTCGCGGCGCTTTATTCAAGAGATAAAGCGCCGCGAGCCG TGAAA-3′ (SEQ ID NO: 3) and 5′-AGCTTTTCACGGCTCGCGGCGCTTTATCTCTTGAATAAAGCGCCGCGAGCC GTGG-3′ (SEQ ID NO: 4) for ACSVL3-3 and 5′-GATCCCGTCTATGGAGTCACTGTGTTCAAGAGACACAGTGACTCCATAGA CGTTA-3′ (SEQ ID NO: 5) and 5′-AGCTTAACGTCTATGGAGTCACTGTGTCTCTTGAACACAGTGACTCCATAG ACGG-3′ (SEQ ID NO: 6) for ACSVL3-4 were annealed and cloned into linearized pSilencer™ 4.1-CMV hygro vector (Applied Biosystems/Ambion). The targeted sequences are underlined. For controls, a pSilencer vector that expresses shRNA with a scrambled sequence not present in human or mouse genomes (supplied by Ambion) was used. U87 cells were transfected with control, ACSVL3-3, ACSVL3-4, or ACSVL3-3 plus ACSVL3-4 plasmids by electroporation using a BTX ECM 600 electroporator. 24 hrs following electroporation, hygromycin (200 μg/ml) was added to the culture medium and antibiotic-resistant clones were selected and analyzed for ASCVL3 KD by immunofluorescence and Western blot. A similar strategy was used to produce stable knockdown of endogenous human FATP4 (SLC27A4; Gene ID 10999) and ACSF2 (Gene ID 80221). The sequence targeted in FATP4 cDNA was 5′-GGTGGGATTCTCCCTGTTG-3′ (SEQ ID NO: 7) (bp 72 to 90 of the coding sequence), and the sequence targeted in ACSF2 cDNA was 5′-GCGAGCCATGGCTGTCTAC-3′ (SEQ ID NO: 8) (bp −7 to +12 of the coding sequence).

Immunohistochemistry, immunofluorescence, and Western blotting. Immunohistochemical staining for ACSVL3 and ACSBG1, and immunofluorescence analysis of ACSVL3, were performed using affinity-purified antibodies as described (9, 15). ACSVL3 was detected on Western blots with SuperSignal West Pico chemiluminescent substrate (Pierce Biotechnology, Rockford Ill.). Total and phospho-Akt (ser473) were detected with antibodies from BD Biosciences (San Jose Calif.) and Cell Signaling Technology (Danvers Mass.), respectively, and quantitated with the LiCOR Odyssey dual wavelength infrared system. Protein was determined by the method of Lowry et al. (16).

Subcutaneous and intracranial xenograft mouse models. All animal protocols were approved by the Johns Hopkins University School of Medicine Animal Care and Use Committee. In vivo tumorigenesis of control and ACSVL3-3 knockdown U87 cells was assessed in 4-6 week-old female mice as previously described (13, 17). For subcutaneous (s.c.) xenografts, NIH III Xid/Beige/Nude mice (National Cancer Institute, Frederick, Md.) were injected with 4×10⁶ cells in 0.1 ml of phosphate-buffered saline (PBS) in the dorsal areas. Tumor growth was measured every 3-4 days using calipers, and tumor sizes were estimated by the formula: volume=(length×width²)/2. For the experiment shown in FIG. 2C, when the tumor size reached ˜300 mm³, the mice were randomly divided into groups (n=6 per group) and injected with the neutralizing anti-HGF mAb L2G7 or control 5G8 monoclonal antibody (mAb) (100 μg/20 g body wt) i.p. twice weekly in a volume of 0.1 ml PBS (17). For orthotopic xenografts, 10⁵ cells in 5 μl PBS were injected unilaterally into the caudate/putamen of C.B-17 Scid/Beige mice (National Cancer Institute, Frederick, Md.) under stereotactic control. Mice were sacrificed 26 days post-injection. Brains were perfusion-fixed and hematoxylin/eosin-stained cryostat sections were used to calculate tumor size by computer-based morphometrics as previously described (18).

Acyl-CoA synthetase assays. Activation of [1-¹⁴C]palmitate (C16:0) or [1-¹⁴C]lignocerate (C24:0) (Moravek Biochemicals, Brea Calif.) to their CoA derivatives was measured in frozen/thawed cell suspensions or post-nuclear supernatants of xenograft tissue homogenates as previously described (9, 19). Assays contained 15 or 60 μg cell or tissue protein for C16:0 and C24:0 fatty acid substrates, respectively.

Results

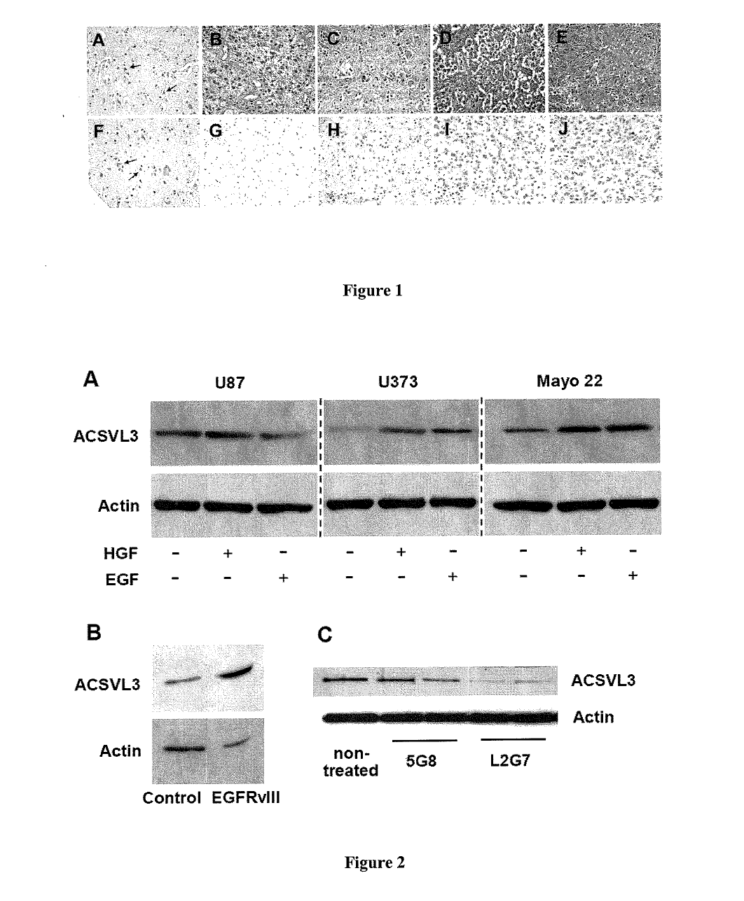

ACSVL3 protein expression is low in normal human brain but increased in malignant gliomas. Immunohistochemical staining of ACSVL3 in adult mouse brain revealed low expression that was limited to neurons; little or no protein was detected in glia (9). A similar staining pattern was found in normal adult human brain (FIG. 1A). In contrast, all tumors examined on a tissue array containing 79 different human gliomas showed increased ACSVL3 expression (FIG. 1B-E), with ˜80% exhibiting robust expression. Gliomas represented on the array included astrocytoma (FIG. 1B), oligodendroglioma (FIG. 1C), anaplastic astrocytoma (FIG. 1D), glioblastoma multiforme (FIG. 1E), and tumors of mixed etiology. The specificity of ACSVL3 overexpression in human glioma specimens was evidenced by the finding that ACSBG1, an ACS expressed in adult mouse neurons (15) and in neurons of normal human brain (FIG. 1F), was not detected in any tumor (FIG. 1G-J).

ACSVL3 expression correlates with malignant phenotype in human glioma cells in vitro and in xenografts. ACSVL3 was readily detectable in established human glioma cell lines and in human glioma cells maintained as xenografts (FIG. 2A). The highly tumorigenic U87 human glioblastoma cell line and cells derived from tumorigenic primary glioblastoma xenografts (Mayo 22) were found to express ACSVL3 at high levels (FIG. 2A). Less tumorigenic U373 glioma cells (13) also expressed ACSVL3 but at lower levels than either U87 or Mayo 22 cells (FIG. 2A). Hepatocyte growth factor (HGF), which enhances glioma cell anchorage independent growth and tumorigenicity was found to induce ACSVL3 expression levels in U373 and Mayo 22 cells (FIG. 2A). Treatment of these cells with the EGFR ligand, epidermal growth factor (EGF), also increased ACSVL3 expression (FIG. 2A). While ACSVL3 expression in U87 cells was not increased above its high basal expression levels by HGF or EGF (FIG. 2A), U87 cells engineered to express the constitutively active EGFR deletion mutant EGFRvIII had considerably more ACSVL3 expression when compared to control-transfected cells (FIG. 2B).

To assess the role of HGF in ACSVL3 expression in vivo, mice bearing pre-established U87 xenografts were treated with neutralizing anti-HGF mAb (L2G7) under conditions that inhibit HGF:c-Met pathway activation and tumor growth (17). Control and anti-HGF treated tumors were resected and tumor extracts assessed for ACSVL3 expression. HGF:c-Met pathway inhibition led to reduced ACSVL3 expression (FIG. 2C) concurrent with tumor growth inhibition. Thus, ACSVL3 is expressed by human glioma cell lines; activation of multiple oncogenic RTK pathways induces ACSVL3 expression, and inhibiting oncogenic RTK signaling inhibits ACSVL3 expression in human glioma xenografts.

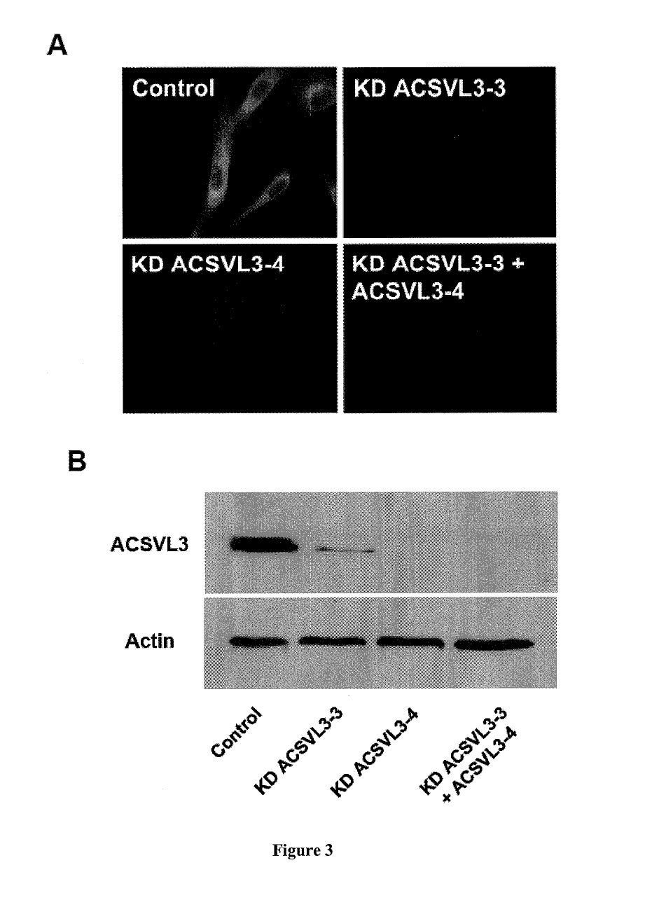

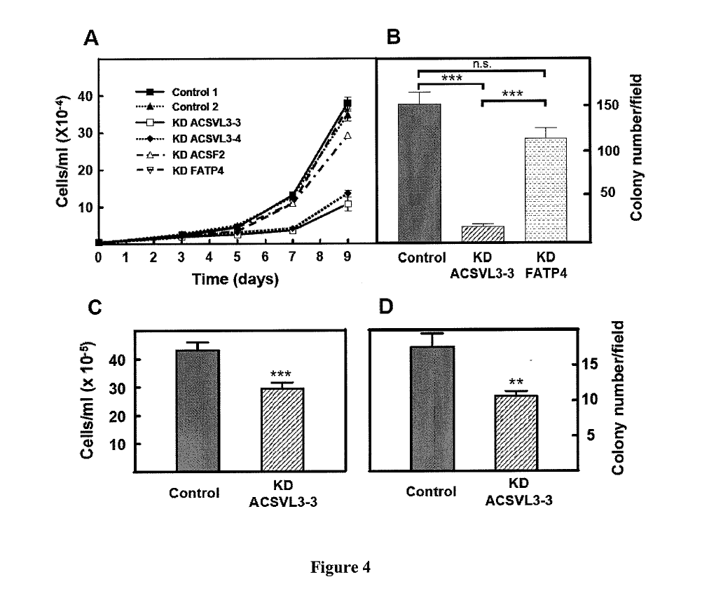

Knockdown of ACSVL3 inhibits glioma cell growth and tumorigenicity. We produced clonal lines of U87 cells in which expression of ACSVL3 was stably knocked down by RNA interference. The two short hairpin-producing plasmids used to construct knockdown (KD) lines, ACSVL3-3 and ACSVL3-4, targeted different regions of ACSVL3 mRNA (see Methods for details). Six clonal lines had nearly undetectable levels of ACSVL3, as judged by indirect immunofluorescence and Western blot. A control and three representative ACSVL3 KD lines are shown in FIG. 3. ACSVL3 KD was further verified by measuring ACS enzyme activity using [1-¹⁴C]FA substrates, as described in Methods; U87 cells lacking ACSVL3 had decreased ability to activate either long-chain or very long-chain FAs relative to control cells (data not shown). To determine the effect of ACSVL3 KD on cellular phenotype, adherent cell growth rates and anchorage-independent clonogenic growth were assessed. ACSVL3 knockdown inhibited anchorage-dependent growth by 33% (FIG. 4A, p<0.001, day 9). This ACSVL3 knockdown also inhibited clonogenicity in soft agar by 87% (FIG. 4B, p<0.001).

ACSVL3 expression was also inhibited in tumor cells derived from Mayo-22 glioblastoma xenografts using ACSVL3-specific siRNA. Anchorage-dependent growth of Mayo 22 cells for seven days post-transfection was decreased by 31% (FIG. 4C; p<0.001), and colony formation in soft agar was reduced by 40% (p<0.01) compared to cells treated with control RNA (FIG. 4D). DNA synthesis, as measured by tritiated thymidine incorporation three days post-transfection, was reduced by 70-85% (p<0.001) in ACSVL3 KD Mayo-22 cells (not shown).

We examined the effects of inhibiting two other ASC family members on glioma cell growth. FATP4 (SLC27A4), an ACSVL3 family member (20), and ACSF2, which activates medium-chain FAs (8); were both expressed by U87 cells under basal conditions (data not shown). Knockdown of either FATP4 or ACSF2 did not appreciably affect U87 cell anchorage-dependent growth (FIG. 4A; p>0.05) or clonogenic growth in soft agar (FIG. 4B; p>0.05). Taken together, these findings demonstrate that ACSVL3 expression is specifically required to support glioma cell growth in vitro.

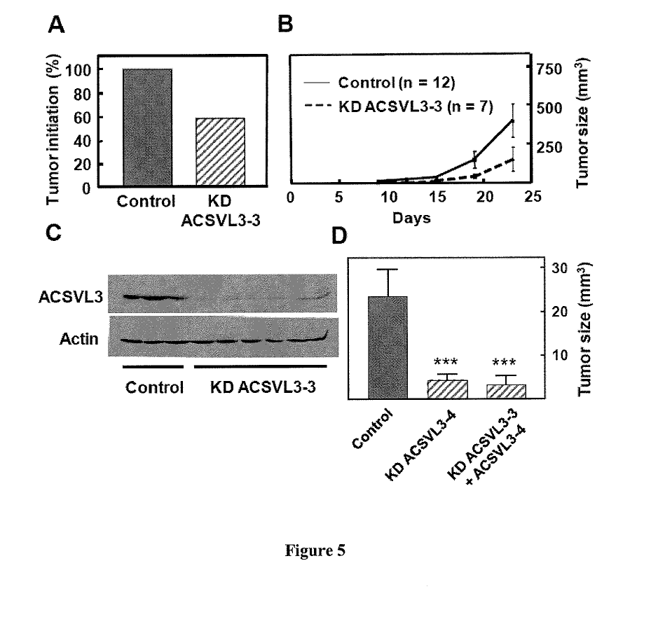

To investigate the effects of ACSVL3 KD on glioma cell tumorigenicity, control-transfected and stable ACSVL3-KD U87 cells were implanted s.c. and tumor growth evaluated. Control-transfected U87 cells generated tumors at all implantation sites (12/12) and ACSVL3-KD cells generated palpable tumors only 58% of the time (7/12) (FIG. 5A). No evidence of tumors was found at the other 5 sites when mice were sacrificed 26 days post-injection. For mice in which ACSVL3-depleted tumors developed, the average xenograft growth rate was reduced by ˜60% (p<0.001, day 23) as assessed by serial caliper measurements (FIG. 5B). ACSVL3 KD tumors resected on post-implantation day 26 weighed 64% less than control xenografts (2.1±0.5 g vs. 0.8±0.4 g, respectively; p<0.001). Western blot analysis verified that ACSVL3 levels were inhibited in ACSVL3 KD tumors compared to control tumors (FIG. 5C), and tumor homogenates had reduced ACS enzyme activity when activation of radiolabeled long-chain and very long-chain FAs was measured as described in Methods (data not shown). ACSVL3 knockdown similarly inhibited the growth of orthotopic glioma xenografts. Orthotopic xenografts were generated by implanting control-transfected or ACSVL3-KD U87 cells to the right caudate/putamen. Mice implanted with control U87 cells developed large (23.5±6.2 mm³) tumors (FIG. 5D). In contrast, xenografts produced by ACSVL3-3 KD or ACSVL3-3+ACSVL3-4 KD U87 cells were over 80% smaller than control tumors (FIG. 5D; p<0.001).

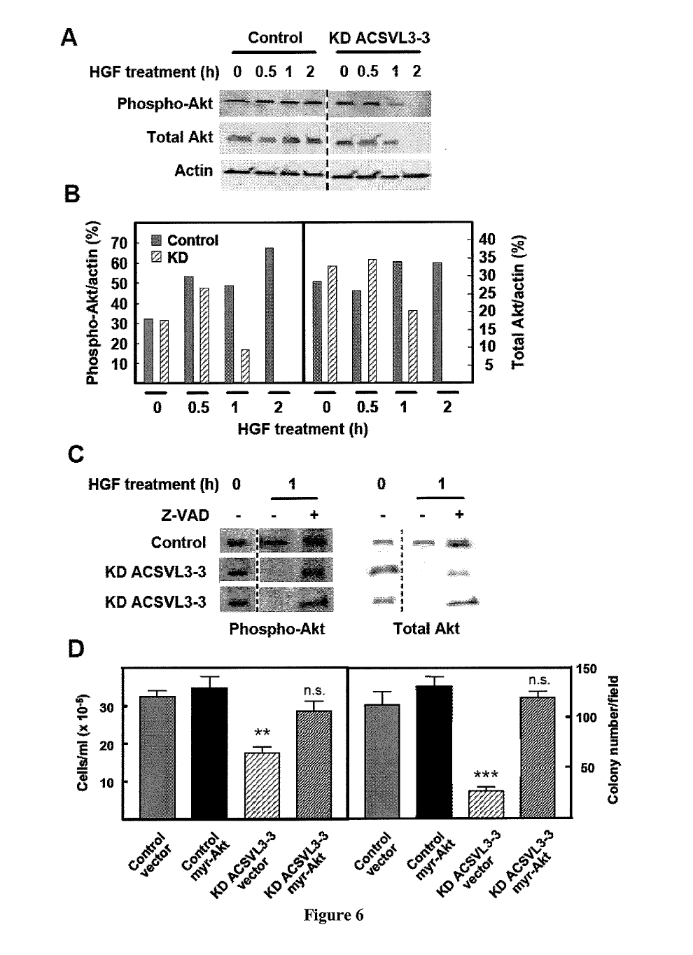

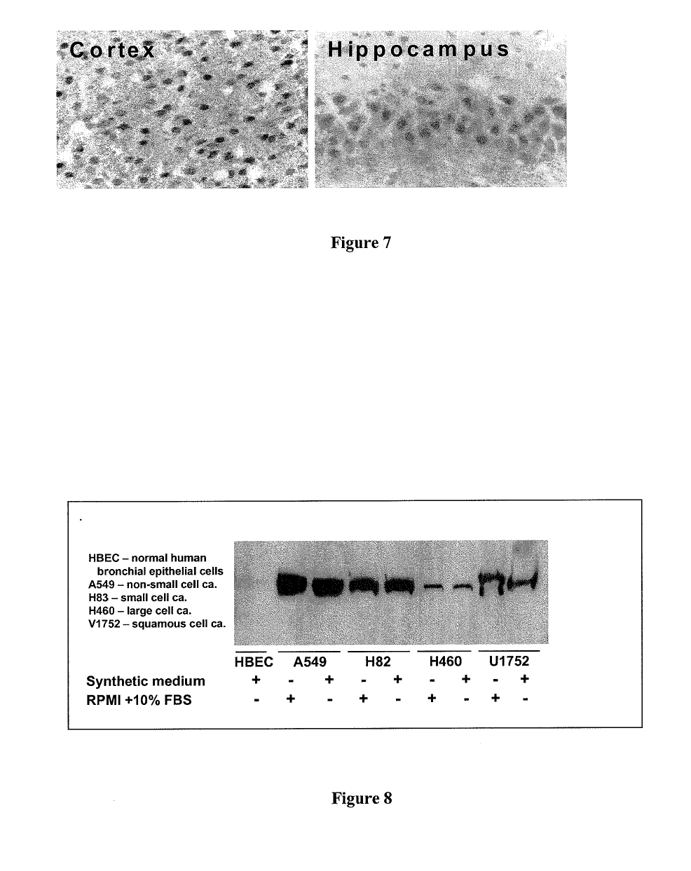

Aberrant Akt signaling results from ACSVL3 knockdown. We tested whether the tumor suppressing effects of ACSVL3 knockdown can result from defects in lipid-dependent oncogenic signaling pathways. RTK signaling pathways support glioma cell growth in vitro and tumorigenicity in vivo (21, 22). Phosphatidylinositol-3 kinase (PI3K) activation by RTKs plays a prominent role in maintaining the malignant glioma phenotype and a prominent downstream target of PI3K is Akt, whose activation requires recruitment by membrane phospholipids. We examined the effect of ACSVL3 knockdown on RTK-induced Akt activation. Akt phosphorylation (Ser-473) in response to HGF (20 ng/ml) increased rapidly and remained elevated for up to two hours in control-transfected U87 glioma cells (FIGS. 6A & B). In ACSVL3-3 KD cells, HGF similarly induced Akt phosphorylation at 30 min, but both total Akt and phospho-Akt levels decreased at 1 hr, and were undetectable at 2 hrs (FIGS. 6A & B). The disappearance of Akt was transient. Phosphorylated Akt and total Akt was detectable again 4 hrs after HGF stimulation and levels returned to baseline by 24 hrs (not shown). Stimulating cells with epidermal growth factor induced a similar Akt response in ACSVL3-3 KD cells (not shown). Akt stability has been shown to be regulated, in part, by caspase-dependent proteolysis (23). Control and ACSVL3 KD U87 cells were treated with HGF in the presence and absence of cell permeable pan-caspase inhibitor, Z-VAD-FMK. Caspase inhibition was found to normalized the Akt response to HGF stimulation in ACSVL3 KD cells (FIG. 6C).

We tested whether Akt instability contributes to the diminished malignant phenotype of ACSVL3 knockdown glioma cells, then constitutive Akt activation should reverse the effects of ACSVL3 knockdown on glioma cell growth. Thus, the effects of constitutively active myr-Akt on the growth of control and ACSVL3 KD cells were examined. Vector-transfected ACSVL3-KD U87 cells displayed significantly diminished anchorage-dependent (FIG. 6D, left panel; p<0.01) and anchorage-independent (FIG. 6D, right panel; p<0.001) growth as compared to control U87 cells, consistent with the results shown in FIG. 4. In contrast, both anchorage-dependent and anchorage-independent growth rates were restored to near normal by myr-Akt (p>0.05 for control myr-Akt vs. KD ACSVL3-3 myr-Akt). Myr-Akt had no appreciable effect on the anchorage-dependent or anchorage-independent growth (p>0.05) of control U87 cells.

### Example 2

Many cancer therapies take advantage of the fact that cancer cells grow and divide more rapidly than normal cells, and thus target the cell cycle. More modern cancer therapeutic approaches target aspects of oncogenic cell signaling pathways, such as growth factor receptors. Targeting metabolic pathways in cancer is a more novel approach to therapy. There has been some recent interest in targeting lipid metabolism in cancer. This stems from the observation that an enzyme of de novo fatty acid synthesis (fatty acid synthase, or FASN) is expressed at high levels in many human tumors. Because elevated FASN is associated with poor prognosis, this enzyme is considered a viable cancer target. Experiments were conducted to elucidate the relation of ACSVL3 to malignancy.

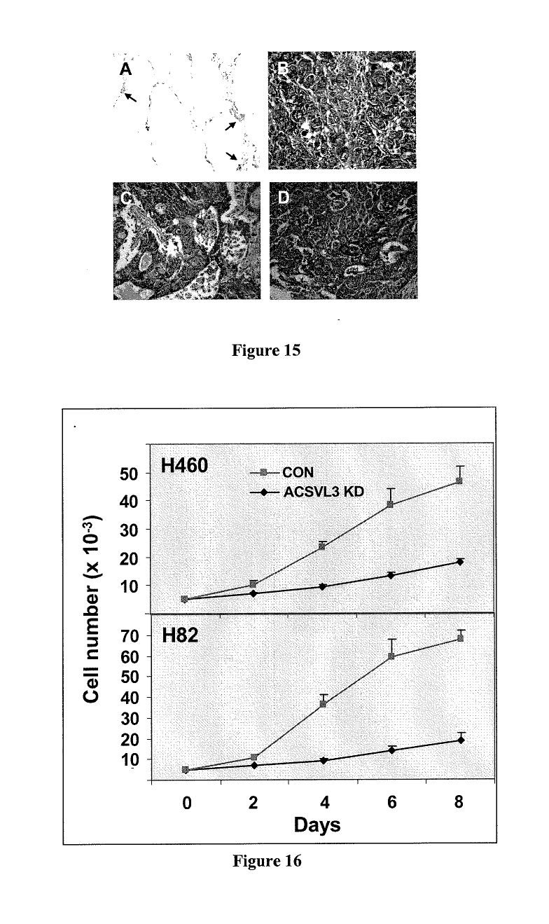

A tissue array containing samples from 69 lung tumors and 3 sections of normal lung was examined for ACSVL3 expression by immunohistochemistry (FIG. 15). The presence of ACSVL3 is indicated by the brown stain. The slide was counterstained with hematoxylin (blue stain). Normal lung showed weak ACSVL3 staining (FIG. 15 A, arrows). Three representative lung tumors are shown in FIG. 15 B-D. This immunohistochemical analysis revealed robust ACSVL3 expression in all tumors, even those of non-epithelial cell origin (e.g. alveolar cell tumors).

ACSVL3 expression correlated with malignancy in human lung cancer cell lines. By Western blot, normal human bronchial epithelial cells (HBEC) in culture had nearly undetectable levels of ACSVL3 (FIG. 8). In contrast, several human lung cancer cell lines (non-small cell, small cell, large cell, and squamous cell origin) showed robust ACSVL3 expression (FIG. 8). A concern was that increases in ACSVL3 were simply the result of the rapid growth rate of tumor cell lines, as HBEC are cultured in synthetic medium lacking serum and grow much slower that the cancer lines, which are grown in RPMI 1760+10% fetal bovine serum. When cancer cell lines were cultured in the same serum-free media used to culture HBEC for at least 2 generations, the cancer cell growth rates were substantially decreased. Despite the much slower growth rate, the level of cancer cell ACSVL3 on Western blots was not changed (FIG. 8), suggesting that the increase in ACSVL3 expression was not simply due to increased cell cycle activity in the cancer cells.

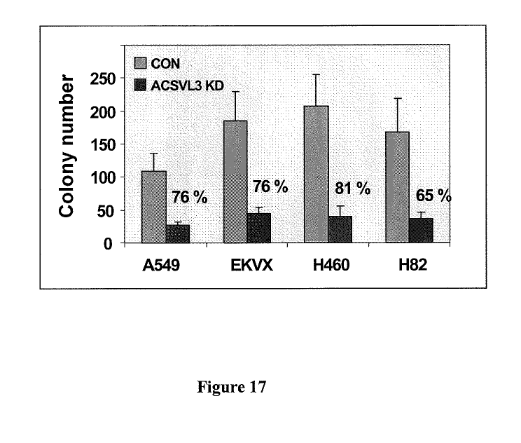

ACSVL3 gene expression knockdown diminishes lung tumor cell proliferation rates and anchorage-independent growth. Based on the RNA interference studies with human glioblastoma cells, we used short hairpin RNAs (shRNA) to produce stable ACSVL3 knockdown in several lung tumor cell lines. Clones with stable ACSVL3 knockdown, and appropriate control lines, were selected using hygromycin resistance. We measured the growth rate for H460 and H82 lung tumor cell lines and found that to be reduced to less than one-third of the growth rate of control tumor cells (FIG. 16). We then investigated anchorage-independent growth of several lung tumor cell lines, and found that deficiency of ACSVL3 reduced significantly their ability to form colonies in soft agar (FIG. 17).

Lipid metabolism studies in control and ACSVL3 knockdown U87 cells and xenografts. Like all other ACSs, ACSVL3 activates fatty acids for downstream lipid metabolic processes as described above. U87 glioblastoma cells lacking ACSVL3 had decreased capacity to activate several fatty acid species (Table 1). Furthermore, the fatty acid profile of ACSVL3-deficient U87 cells was significantly different from that of control U87 cells. Total cellular lipids were hydrolyzed, releasing their constituent FAs, which were then derivatized and quantitated by gas chromatography (GC). Results of three independent analyses are shown in Table 2. Based on our experience, the most interesting differences are (i) the increased levels of saturated and monounsaturated very long-chain FAs (C26:0, C28:0, C26:1), (ii) the increased levels of ω7 FAs, and (iii) decreased levels of to ω3 FAs.

TABLE 1
FA substrate preference of ACSVL3. Loss of ability to activate
a specific FA in ACSVL3 KD cells indicates specificity for that FA.
Cultured U87 cells and homogenates of xenografts produced from
U87 cells were assayed for ACS activity with the indicated FA
substrate. Mean ± SD is shown; n = 3-4.

| | | ACSVL3- | | ACSVL3- |
|---|---|---|---|---|
| | Control | deficient | | deficient |
| | (nmol/20 min/mg protein) | | p-value | (% of control) |
| Cultured | | | | |
| U87 cells: | | | | |
| C8:0 | 0.28 ± 0.01 | 0.25 ± 0.03 | n.s. | 89 |
| C12:0 | 10.5 ± 0.8 | 9.6 ± 0.6 | n.s. | 91 |
| C16:0 | 44.2 ± 1.9 | 29.1 ± 3.2 | <0.0001 | 66 |
| C18:1 | 6.3 ± 0.1 | 3.7 ± 0.3 | <0.0001 | 59 |
| C24:0 | 2.5 ± 0.1 | 1.9 ± 0.2 | <0.002 | 76 |
| U87 xenograft | | | | |
| tissue: | | | | |
| C16:0 | 37.4 ± 2.5 | 23.2 ± 1.9 | <0.0001 | 62 |
| C18:1 | 7.2 ± 0.6 | 4.3 ± 0.5 | <0.0001 | 60 |
| C24:0 | 2.5 ± 0.2 | 1.8 ± 0.1 | <0.0001 | 72 |

TABLE 2
FA analysis of control and ACSVL3 KD U87 cells. Cells grown
to confluence were harvested, subjected to acid methanolysis,
and total fatty acyls analyzed by GC/MS. Mean ± SD for three
independent analyses is shown. P-values were calculated using
an unpaired, two-tailed t-test.

| | | ACSVL3- | | ACSVL3- |
|---|---|---|---|---|
| | Control | deficient | | deficient |
| | (% of total fatty acids) | | p-value | (% of control) |
| All saturated | 34.5 ± 1.7 | 30.7 ± 0.6 | <0.05 | 89 |
| fatty acids | | | | |
| C16:0 | 17.8 ± 0.5 | 15.5 ± 0.6 | <0.01 | 87 |
| C18:0 | 10.5 ± 2.9 | 8.8 ± 0.4 | n.s. | 84 |
| C24:0 | 2.05 ± 0.80 | 1.89 ± 0.21 | n.s. | 92 |
| C26:0 | 0.28 ± 0.16 | 0.60 ± 0.07 | <0.05 | 214 |
| C28:0 | 0.02 ± 0.01 | 0.09 ± 0.02 | <0.01 | 450 |
| All ω9 fatty acids | 31.2 ± 1.0 | 33.6 ± 0.9 | <0.05 | 108 |
| C16:1 (ω9) | 1.29 ± 0.19 | 0.93 ± 0.13 | n.s. | 72 |
| C18:1 (ω9) | 25.1 ± 0.5 | 26.5 ± 0.7 | n.s. | 106 |
| C24:1 (ω9) | 1.56 ± 0.20 | 1.90 ± 0.10 | n.s. | 122 |
| C26:1 (ω9) | 0.37 ± 0.18 | 0.75 ± 0.13 | <0.05 | 203 |
| All ω7 fatty acids | 12.5 ± 2.7 | 19.5 ± 1.28 | <0.02 | 156 |
| C16:1 (ω7) | 2.99 ± 1.64 | 4.95 ± 0.89 | n.s. | 166 |
| C18:1 (ω7) | 9.4 ± 1.1 | 14.4 ± 0.4 | <0.002 | 153 |
| All ω6 fatty acids | 13.2 ± 3.4 | 10.0 ± 3.2 | n.s. | 76 |
| C20:3 (ω6) | 1.51 ± 0.45 | 0.85 ± 0.15 | n.s. | 56 |
| C20:4 (ω6) | 6.43 ± 1.90 | 4.34 ± 1.78 | n.s. | 67 |
| C22:4 (ω6) | 1.81 ± 0.39 | 1.37 ± 0.52 | n.s. | 76 |
| All ω3 fatty acids | 7.37 ± 1.19 | 5.28 ± 0.41 | <0.05 | 72 |
| C20:5 (ω3) | 1.12 ± 0.16 | 0.47 ± 0.14 | <0.01 | 42 |
| C22:6 (ω3) | 3.03 ± 0.44 | 2.25 ± 0.13 | <0.05 | 74 |

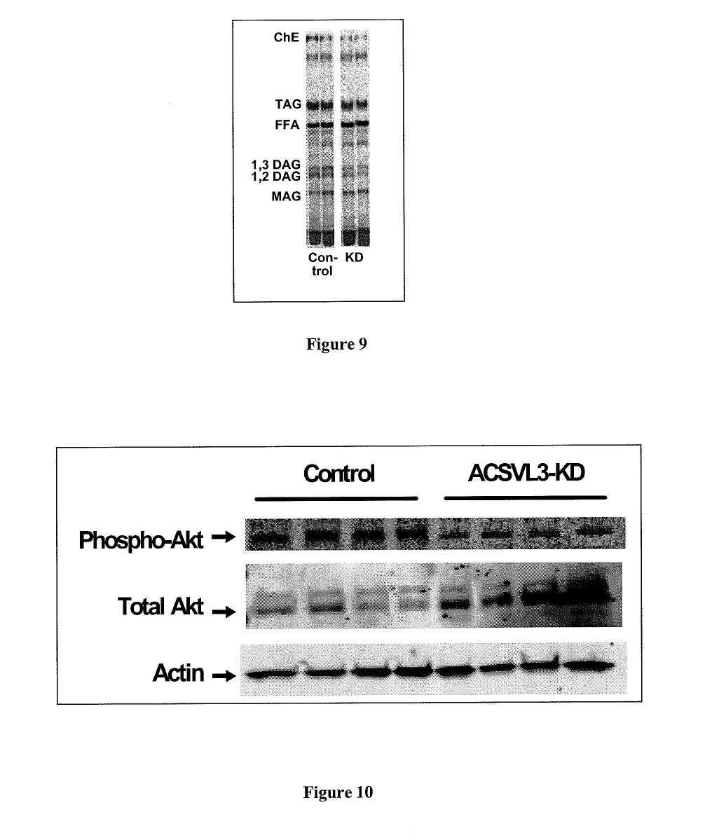

Both ACSVL3-depleted U87 cells (FIG. 9) and homogenates of tumors produced from U87 ACSVL3 KD cells (not shown) had decreased synthesis of the lipid signaling molecule, diacylglycerol (DAG). Synthesis of other neutral lipids and phospholipids were not changed by lack of ACSVL3. Decreased DAG synthesis suggests that ACSVL3 plays a specific role in signaling via phospholipase C-γ.

Summary. The findings show that ACSVL3 is elevated in human lung tumors. ACSVL3 is similarly elevated in human lung tumor cell lines. Studies of human lung cell lines show that decreasing the cellular level of ACSVL3 reduces the malignant phenotype in vitro. Decreasing ACSVL3 affects cellular lipid metabolism, cellular fatty acid profiles, and lipids related to cellular signaling pathways. These observations demonstrate that ACSVL3 is a viable new cancer target.

### Example 3

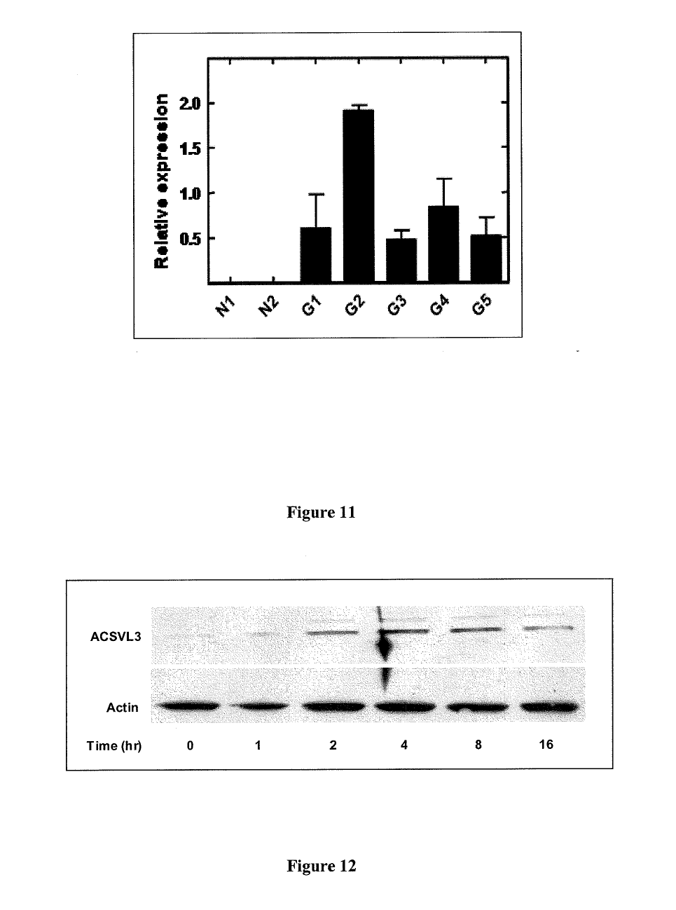

Quantitation of ACSVL3 mRNA in human glioblastomas. To substantiate our observation that ACSVL3 protein is elevated in human glioblastomas, ACSVL3 mRNA levels were quantitated. RNA from normal human brain and glioblastomas was a gift from Dr. Charles Eberhart, Johns Hopkins University School of Medicine, Baltimore, Md. Forward primer 5′-CCCAGAGTTTCTGTGGCTCT-3′ (SEQ ID NO: 9) and reverse primer 5′-GGACACTTCAGCCAGCAAAT-3′ (SEQ ID NO: 10) were designed to amplify a 256 bp fragment of ACSVL3 that spanned the boundary between exons 1 and 2. One-step reverse transcriptase-polymerase chain reaction (RT-PCR) was performed using the iScript SYBR Green RT-PCR kit and the iQ5 Real-Time PCR Detection System (Bio-Rad Laboratories, Hercules, Calif.). Amplification of 18S RNA was performed as a control. Analysis was performed by iQ5 (Bio-Rad) and Prism (GraphPad Software, La Jolla, Calif.) software. The ratio of ACSVL3 to 18S RNA for triplicate determinations was plotted as mean±standard error. ACSVL3 mRNA levels were significantly greater in glioblastomas (G1-G5) than in normal brain (N1, N2) (FIG. 11). For N1 and N2, the ratio was <0.005 and is thus not visible on the scale of the plot.

Further characterization of induction of ACSVL3 expression in U373 cells. Human U373 glioma cells have low ACSVL3 expression unless treated with hepatocyte growth factor (HGF). We investigated the time course of induction by HGF of ACSVL3 protein expression in U373 cells. The Western blot shown in FIG. 12 demonstrates that ACSVL3 protein levels are increased in U373 cells as early as 2 hours following treatment with HGF.

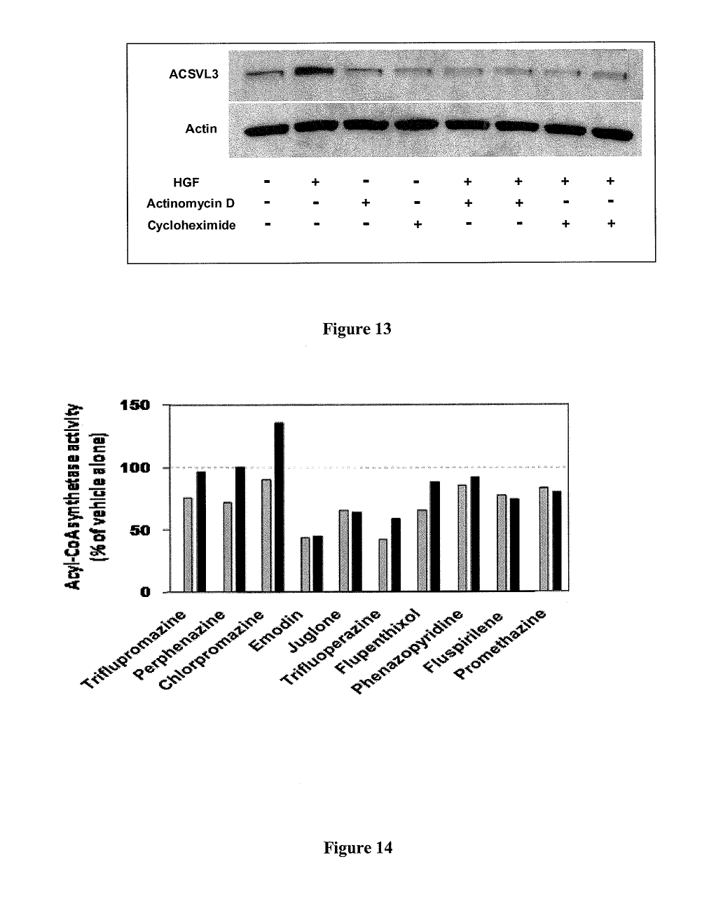

We then investigated whether the increase in ACSVL3 protein in U373 cells was due to increased transcription of the ACSVL3 gene, or increased translation of ACSVL3 mRNA. U373 cells were incubated without or with HGF for 6 hours in the absence and presence of an inhibitor of transcription, actinomycin D, or the protein synthesis (translation) inhibitor, cycloheximide. Actinomycin D (20 μg/ml) and cycloheximide (5 μM) were added to the culture medium 16 and 8 hours, respectively, prior to the addition of HGF. In the absence of HGF, neither actinomycin D nor cycloheximide affected ACSVL3 expression (FIG. 13). In the presence of HGF, both agents blocked the HGF-induced increase in ACSVL3 expression, indicating that HGF acts to promote transcription of the ACSVL3 gene.

Identification and testing of candidate small molecule ACSVL3 inhibitors. ACSVL3 belongs to the very long-chain subfamily of acyl-CoA synthetases. Members of this subfamily are also known as Fatty Acid Transport Proteins. Some, but all FATPs facilitate the cellular uptake of fluorescently labeled fatty acids (Hirsch et al. 1998). Black, DiRusso and colleagues (DiRusso et al. 2005; Li et al. 2005) established a system to identify inhibitors of fluorescent fatty acid uptake in cells expressing FATPs. They first created a mutant yeast strain that lacked two endogenous acyl-CoA synthetases (FAA1p and Fat1 p). They then individually expressed mammalian FATP proteins in this strain, and determined whether the FATP facilitated uptake of the fluorescent fatty acid. FATP1, FATP2 (=ACSVL1), and FATP4 facilitated fatty acid uptake, whereas FATP3 (=ACSVL3), FATP5, and FATP6 did not (DiRusso et al. 2005). Using this concept, these investigators next developed a high-throughput screening method which they used to identify inhibitors of fluorescent fatty acid uptake in mutant yeast cells expressing FATP2. Several compounds were identified using this method, and the results were published (Li et al. 2005).

We have shown that for FATP1, FATP2, and FATP4, it is their inherent acyl-CoA synthetase activity that drives fluorescent fatty acid uptake, and that inhibition of fatty acid uptake reflects inhibition of acyl-CoA synthetase enzymatic activity. As noted, ACSVL3 (=FATP3) did not facilitate the uptake of fluorescent fatty acid; thus, the assay described by Black, DiRusso and colleagues cannot be used directly to identify specific inhibitors of ACSVL3. However, we further conclude that because ACSVL3 and FATP2 (=ACSVL1) are structurally and functionally related acyl-CoA synthetases, small molecule inhibitors of FATP2, or related small molecules, can also inhibit ACSVL3 and/or other acyl-CoA synthetases. We therefore tested certain small molecule compounds identified as inhibitors of FATP2 by Black, DiRusso and colleagues and published in the Journal of Lipid Research (Li et al. 2005) for their ability to inhibit ACSVL3.

For this study, we examined the effects of candidate small molecule inhibitors on the acyl-CoA synthetase activity of control U87 glioblastoma cells and ACSVL3-deficient U87 cells. The 16-carbon fatty acid, palmitic acid, was used as substrate. Drugs were solubilized in dimethylsulfoxide (DMSO, 10 mM) and diluted with phosphate-buffered saline (PBS, 0.8 mM) prior to their inclusion in enzyme assays; the final concentration of drug in the assay was 80 μM. Control incubations contained the vehicle, DMSO diluted in PBS, only. The assay measures the combined enzyme activity of all acyl-CoA synthetases found in U87 cells that are capable of using palmitic acid as substrate. Results of some preliminary studies are shown in FIG. 14. A specific inhibitor of ACSVL3 is one in which there is partial inhibition of total cellular enzyme activity in control U87 cells (grey bars), but not in ACSVL3-deficient U87 cells (black bars). Triflupromazine, perphenizine, and chlorpromazine exhibited specificity for ACSVL3. Drugs that inhibit enzyme activity similarly in both control and ACSVL3-deficient U87 cells, such as emodin, juglone, phenazopyridine, fluspirilene, and promethazine, are likely inhibiting acyl-CoA synthetases other than ACSVL3. Drugs that inhibit both control and ACSVL3-deficient cells, but show greater inhibition in control U87 cells, such as trifluperazine and flupenthixol, are likely inhibiting both ACSVL3 and other acyl-CoA synthetases.

These examples illustrate possible embodiments of the present invention. As one of skill in the art will appreciate, because of the versatility of the methods disclosed herein, they may be used in other similar ways to those described herein. Thus, while the invention has been particularly shown and described with reference to some embodiments thereof, it will be understood by those skilled in the art that they have been presented by way of example only, and not limitation, and various changes in form and details may be made therein without departing from the spirit and scope of the invention. Therefore, the breadth and scope of the present invention should not be limited by any of the above-described embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

### REFERENCE LIST

### Each of which is Specifically Incorporated by Reference Herein

1. Ohgaki, H. and Kleihues, P. Epidemiology and etiology of gliomas. Acta Neuropathol (Berl), 109: 93-108, 2005.
2. Patra, S. K. Dissecting lipid raft facilitated cell signaling pathways in cancer. Biochim Biophys Acta, 1785: 182-206, 2008.
3. Ookhtens, M., Kannan, R., Lyon, I., and Baker, N. Liver and adipose tissue contributions to newly formed fatty acids in an ascites tumor. Am J Physiol, 247: R146-153, 1984.
4. Kuhajda, F. P., Jenner, K., Wood, F. D., Hennigar, R. A., Jacobs, L. B., Dick, J. D., and Pasternack, G. R. Fatty acid synthesis: a potential selective target for antineoplastic therapy. Proc Natl Acad Sci USA, 91: 6379-6383, 1994.
5. Kuhajda, F. P. Fatty-acid synthase and human cancer: new perspectives on its role in tumor biology. Nutrition, 16: 202-208, 2000.
6. Zhao, W., Kridel, S., Thorburn, A., Kooshki, M., Little, J., Hebbar, S., and Robbins, M. Fatty acid synthase: a novel target for antiglioma therapy. Br J Cancer, 95: 869-878, 2006.
7. Watkins, P. A. Fatty acid activation. Prog. Lipid Res., 36: 55-83, 1997.
8. Watkins, P. A., Maiguel, D., Jia, Z., and Pevsner, J. Evidence for 26 distinct acyl-coenzyme A synthetase genes in the human genome. J Lipid Res, 48: 2736-2750, 2007.
9. Pei, Z., Fraisl, P., Berger, J., Jia, Z., Forss-Petter, S., and Watkins, P. A. Mouse very long-chain Acyl-CoA synthetase 3/fatty acid transport protein 3 catalyzes fatty acid activation but not fatty acid transport in MA-10 cells. J Biol Chem, 279: 54454-54462, 2004.
10. Xia, S., Rosen, E. M., and Laterra, J. Sensitization of glioma cells to Fas-dependent apoptosis by chemotherapy-induced oxidative stress. Cancer Res, 65: 5248-5255, 2005.
11. Nishikawa, R., Ji, X. D., Harmon, R. C., Lazar, C. S., Gill, G. N., Cavenee, W. K., and Huang, H. J. A mutant epidermal growth factor receptor common in human glioma confers enhanced tumorigenicity. Proc Natl Acad Sci USA, 91: 7727-7731, 1994.
12. Pandita, A., Aldape, K. D., Zadeh, G., Guha, A., and James, C. D. Contrasting in vivo and in vitro fates of glioblastoma cell subpopulations with amplified EGFR. Genes Chromosomes Cancer, 39: 29-36, 2004.
13. Laterra, J., Rosen, E., Nam, M., Ranganathan, S., Fielding, K., and Johnston, P. Scatter factor/hepatocyte growth factor expression enhances human glioblastoma tumorigenicity and growth. Biochem Biophys Res Commun, 235: 743-747, 1997.
14. Walter, K. A., Hossain, M. A., Luddy, C., Goel, N., Reznik, T. E., and Laterra, J. Scatter factor/hepatocyte growth factor stimulation of glioblastoma cell cycle progression through G(1) is c-Myc dependent and independent of p27 suppression, Cdk2 activation, or E2F1-dependent transcription. Mol Cell Biol, 22: 2703-2715, 2002.
15. Pei, Z., Oey, N. A., Zuidervaart, M. M., Jia, Z., Li, Y., Steinberg, S. J., Smith, K. D., and Watkins, P. A. The acyl-CoA synthetase “bubblegum” (lipidosin): further characterization and role in neuronal fatty acid beta-oxidation. J Biol Chem, 278: 47070-47078, 2003.
16. Lowry, 0. H., Rosebrough, N. J., Farr, A. L., and Randall, R. J. Protein measurement with the Folin phenol reagent. J. Biol. Chem., 193: 265-275, 1951.
17. Kim, K. J., Wang, L., Su, Y. C., Gillespie, G. Y., Salhotra, A., Lal, B., and Laterra, J. Systemic anti-hepatocyte growth factor monoclonal antibody therapy induces the regression of intracranial glioma xenografts. Clin Cancer Res, 12: 1292-1298, 2006.
18. Abounader, R., Lal, B., Luddy, C., Koe, G., Davidson, B., Rosen, E. M., and Laterra, J. In vivo targeting of SF/HGF and c-met expression via U1snRNA/ribozymes inhibits glioma growth and angiogenesis and promotes apoptosis. Faseb J, 16: 108-110., 2002.
19. Jia, Z., Moulson, C. L., Pei, Z., Miner, J. H., and Watkins, P. A. Fatty acid transport protein 4 is the principal very long chain fatty acyl-CoA synthetase in skin fibroblasts. J Biol Chem, 282: 20573-20583, 2007.
20. Watkins, P. A. Very-long-chain Acyl-CoA Synthetases. J Biol Chem, 283: 1773-1777, 2008.
21. Kapoor, G. S. and O'Rourke, D. M. Receptor tyrosine kinase signaling in gliomagenesis: pathobiology and therapeutic approaches. Cancer Biol Ther, 2: 330-342, 2003.
22. Birchmeier, C., Birchmeier, W., Gherardi, E., and Vande Woude, G. F. Met, metastasis, motility and more. Nat Rev Mol Cell Biol, 4: 915-925, 2003.
23. Martin, D., Salinas, M., Fujita, N., Tsuruo, T., and Cuadrado, A. Ceramide and reactive oxygen species generated by H202 induce caspase-3-independent degradation of Akt/protein kinase B. J Biol Chem, 277: 42943-42952, 2002.
24. Kuhajda, F. P. Fatty acid synthase and cancer: new application of an old pathway. Cancer Res, 66: 5977-5980, 2006.
25. Yeh, C. S., Wang, J. Y., Cheng, T. L., Juan, C. H., Wu, C. H., and Lin, S. R. Fatty acid metabolism pathway play an important role in carcinogenesis of human colorectal cancers by Microarray-Bioinformatics analysis. Cancer Lett, 233: 297-308, 2005.
26. Gassler, N., Herr, I., Schneider, A., Penzel, R., Langbein, L., Schirmacher, P., and Kopitz, J. Impaired expression of acyl-CoA synthetase 5 in sporadic colorectal adenocarcinomas. J Pathol, 207: 295-300, 2005.
27. Gassler, N., Yang, S. H., Keith, M., Helmke, B. M., Schirmacher, P., and Obermuller, N. Expression of acyl-CoA synthetase 5 in human endometrium and in endometrioid adenocarcinomas. Histopathology, 47: 501-507, 2005.
28. Yamashita, Y., Kumabe, T., Cho, Y. Y., Watanabe, M., Kawagishi, J., Yoshimoto, T., Fujino, T., Kang, M. J., and Yamamoto, T. T. Fatty acid induced glioma cell growth is mediated by the acyl-CoA synthetase 5 gene located on chromosome 10q25.1-q25.2, a region frequently deleted in malignant gliomas. Oncogene, 19: 5919-5925, 2000.
29. Kim, J. H., Lewin, T. M., and Coleman, R. A. Expression and characterization of recombinant rat Acyl-CoA synthetases 1, 4, and 5. Selective inhibition by triacsin C and thiazolidinediones. J Biol Chem, 276: 24667-24673, 2001.
30. Van Horn, C. G., Caviglia, J. M., Li, L. O., Wang, S., Granger, D. A., and Coleman, R. A. Characterization of Recombinant Long-Chain Rat Acyl-CoA Synthetase Isoforms 3 and 6: Identification of a Novel Variant of Isoform 6. Biochemistry, 44: 1635-1642, 2005.
31. Li, H., Black, P. N., and DiRusso, C. C. A live-cell high-throughput screening assay for identification of fatty acid uptake inhibitors. Anal Biochem, 336: 11-19, 2005.
32. Hall, A. M., Smith, A. J., and Bernlohr, D. A. Characterization of the Acyl CoA synthetase activity of purified murine fatty acid transport protein 1. J Biol Chem, 278: 43008-43013, 2003.
33. Hall, A. M., Wiczer, B. M., Herrmann, T., Stremmel, W., and Bernlohr, D. A. Enzymatic Properties of Purified Murine Fatty Acid Transport Protein 4 and Analysis of Acyl-CoA Synthetase Activities in Tissues from FATP4 Null Mice. J Biol Chem, 280: 11948-11954, 2005.
34. Pei, Z., Jia, Z., and Watkins, P. A. The second member of the human and murine bubblegum family is a testis- and brainstem-specific acyl-CoA synthetase. J Biol Chem, 281: 6632-6641, 2006.
35. Mashima, T., Oh-hara, T., Sato, S., Mochizuki, M., Sugimoto, Y., Yamazaki, K., Hamada, J., Tada, M., Moriuchi, T., Ishikawa, Y., Kato, Y., Tomoda, H., Yamori, T., and Tsuruo, T. p53-defective tumors with a functional apoptosome-mediated pathway: a new therapeutic target. J Natl Cancer Inst, 97: 765-777, 2005.
36. Wong, A. J., Bigner, S. H., Bigner, D. D., Kinzler, K. W., Hamilton, S. R., and Vogelstein, B. Increased expression of the epidermal growth factor receptor gene in malignant gliomas is invariably associated with gene amplification. Proc Natl Acad Sci USA, 84: 6899-6903, 1987.
37. Simmons, M. L., Lamborn, K. R., Takahashi, M., Chen, P., Israel, M. A., Berger, M. S., Godfrey, T., Nigro, J., Prados, M., Chang, S., Barker, F. G., 2nd, and Aldape, K. Analysis of complex relationships between age, p53, epidermal growth factor receptor, and survival in glioblastoma patients. Cancer Res, 61: 1122-1128, 2001.
38. Hermanson, M., Funa, K., Hartman, M., Claesson-Welsh, L., Heldin, C. H., Westermark, B., and Nister, M. Platelet-derived growth factor and its receptors in human glioma tissue: expression of messenger RNA and protein suggests the presence of autocrine and paracrine loops. Cancer Res, 52: 3213-3219, 1992.
39. Abounader, R. and Laterra, J. Scatter factor/hepatocyte growth factor in brain tumor growth and angiogenesis. Neuro-oncol, 7: 436-451, 2005.
40. Bowers, D. C., Fan, S., Walter, K. A., Abounader, R., Williams, J. A., Rosen, E. M., and Laterra, J. Scatter factor/hepatocyte growth factor protects against cytotoxic death in human glioblastoma via phosphatidylinositol 3-kinase- and AKT-dependent pathways. Cancer Res, 60: 4277-4283, 2000.
41. Rokudai, S., Fujita, N., Hashimoto, Y., and Tsuruo, T. Cleavage and inactivation of antiapoptotic Akt/PKB by caspases during apoptosis. J Cell Physiol, 182: 290-296, 2000.
42. Lee, J. H., Shin, S. H., Kang, S., Lee, Y. S., and Bae, S. A novel activation-induced suicidal degradation mechanism for Akt by selenium. Int J Mol Med, 21: 91-97, 2008.
43. Abounader, R., Ranganathan, S., Lal, B., Fielding, K., Book, A., Dietz, H., Burger, P. and Laterra, J. (1999) Reversion of human glioblastoma malignancy by U1 small nuclear RNA/ribozyme targeting of scatter factor/hepatocyte growth factor and c-met expression. J Natl Cancer Inst 91, 1548-56.
44. Guerin, C., Luddy, C., Abounader, R., Lal, B. and Laterra, J. (2000) Glioma inhibition by HGF/NK2, an antagonist of scatter factor/hepatocyte growth factor. Biochem Biophys Res Commun 273, 287-93.
45. Hirsch, D., Stahl, A. and Lodish, H. F. (1998) A family of fatty acid transporters conserved from mycobacterium to man. Proc Natl Acad Sci USA 95, 8625-9.
46. Li, Y., Lal, B., Kwon, S., Fan, X., Saldanha, U., Reznik, T. E., Kuchner, E. B., Eberhart, C., Laterra, J. and Abounader, R. (2005) The scatter factor/hepatocyte growth factor: c-met pathway in human embryonal central nervous system tumor malignancy. Cancer Res 65, 9355-62.
47. Watkins, P. A., Pevsner, J. and Steinberg, S. J. (1999) Human very long-chain acyl-CoA synthetase and two human homologs: initial characterization and relationship to fatty acid transport protein. Prostaglandins Leukot Essent Fatty Acids 60, 323-8.

We claim:

1. A method of reducing cancer cell proliferation comprising administering an effective amount of a compound that inhibits the activity of ACSVL3 to a cancer cell expressing ACSVL3.

2. The method of claim 1, wherein the compound is selected from the group consisting of siRNA, flupenthixol, triflupromazine, perphenizine, and chlorpromazine.

3. The method of claim 1, wherein the cells are malignant cancer cells.

4. The method of claim 1, wherein the cancer cells are glioma cells or human lung cancer cells.

5. The method of claim 1, wherein the reduction of cellular proliferation is about 20% to about 90%.

6. The method of claim 1, wherein the compound is administered to a mammal in need of treatment for lung cancer or brain cancer.

7. The method of claim 6, wherein the mammal is a human.

8. The method of claim 1, wherein the method further inhibits c-Met signaling.

9. The method of claim 1, wherein the tumor cells exhibit increased ACSVL3 expression relative to a normal cell.

10. A method of treating cancer comprising administering a therapeutically effective amount of a compound that inhibits the activity of ACSVL3 to a cancer cell expressing ACSVL3.

11. Use of a therapeutically effective amount of a compound selected from the group consisting of siRNA, flupenthixol, triflupromazine, perphenizine, and chlorpromazine to treat cancer.

12. An siRNA comprising a sequence encoding at least one short hairpin RNA(shRNA) that can inhibit the production of at least one Acyl-Co A synthetase, very long chain family member (ACSVL) when contacted with mammalian cells that express at least one ACSVL.

13. The siRNA of claim 12, wherein the sequence is ACSVL3-3 or ACSVL3-4.
